(19) United States
(12) Patent Application Publication
(10) Pub. No.: US 2018/0318082 A1
(43) Pub. Date: Nov. 8, 2018

(54) PAPILLARY MUSCLE BINDING

(71) Applicant: Edwards Lifesciences Corporation, Irvine, CA (US)

(72) Inventor: Boaz Manash, Givat Ada (IL)

(21) Appl. No.: 15/960,341

(22) Filed: Apr. 23, 2018

Related U.S. Application Data
(60) Provisional application No. 62/501,980, filed on May 5, 2017.

(51) Int. Cl. A61F 2/24

(52) U.S. Cl. CPC A61F 2/2457; A61F 2/2466; A61F 2250/0012; A61F 2220/0008; A61F 2210/0014

(57) ABSTRACT

A method for improving leaflet prolapse and/or valve regurgitation associated with a heart valve involves delivering a catheter into a ventricle of a heart, attaching a cord to a first papillary muscle disposed in the ventricle, the first papillary muscle being connected to a first leaflet of an atrioventricular heart valve, attaching the cord to a second papillary muscle, the second papillary muscle being connected to a second leaflet of the heart valve, secure first and second portions of the cord in a fixed relative position to form a loop attached to both the first and second papillary muscles, and releasing the cord from the catheter.

### CROSS-REFERENCE TO RELATED APPLICATIONS

This application claims the benefit of U.S. Application No. 62/501,980, filed May 5, 2017, incorporated herein by reference.

### BACKGROUND

### Field

The present disclosure generally relates to the field of valve correction.

### Description of Related Art

Heart valve dysfunction can result in regurgitation and other complications due to valve prolapse from failure of valve leaflets to properly coapt. For atrioventricular valves, papillary muscle position can affect the ability of valve leaflets to function properly.

### SUMMARY

In some implementations, the present disclosure relates to papillary muscle binding and/or reshaping methods, systems, and devices that may improve heart valve performance by reducing regurgitation and/or leaflet prolapse. In accordance with some embodiments, a method for treating heart valves comprises delivering a catheter into a ventricle of a heart, attaching a cord to a first papillary muscle disposed in the ventricle, the first papillary muscle being connected to a first leaflet of an atrioventricular heart valve, attaching the cord to a second papillary muscle, the second papillary muscle being connected to a second leaflet of the heart valve, securing first and second portions of the cord in a fixed relative position to form a loop attached to both the first and second papillary muscles, and releasing the cord from the catheter. Performing the method improves at least one of prolapse of the first and second leaflets and regurgitation of the heart valve. In some embodiments, the ventricle may be a left ventricle of the heart and the heart valve may be a mitral valve.

In certain embodiments, attaching the cord to the first papillary muscle comprises puncturing the first papillary muscle and threading a distal end of the cord through at least a portion of the first papillary muscle. For example, puncturing the first papillary muscle may comprise introducing a flexible needle from the catheter and puncturing the first papillary muscle using the flexible needle.

Securing the first and second portions of the cord in a fixed relative position may comprise introducing the first and second portions of the cord into a locker device, and locking the locker device to secure the first and second portions of the cord. In certain embodiments, the locker device is a winch locker. The method may further comprise winding one or more of the first and second portions of the cord on a spool component of the winch locker to achieve a desired tension in the cord. In certain embodiments, the locker device is a clamp, wherein locking the locker device comprises closing first and second portions of the clamp over the first and second portions of the cord.

The method may further comprise, after said attaching the cord to the second papillary muscle, determining whether function of the heart valve is adequate, and when it is determined that the function of the heart valve is not adequate, adjusting a tension of the cord prior to said releasing the cord from the catheter. In certain embodiments, determining whether function of the heart valve is adequate is performed at least part using echocardiography. Determining whether function of the heart valve is adequate may comprise determining whether the first papillary muscle has moved to a desirable position. Determining whether function of the heart valve is adequate may comprises determining whether regurgitation of the heart valve has been sufficiently reduced. In certain embodiments, determining whether function of the heart valve is adequate comprises determining whether a position of the first or second leaflet has moved to a desirable position.

The method may further comprise adjusting a tension of the cord prior to securing the first and second portions of the cord. Adjusting the tension in the cord may comprise cinching the cord using a portion of the catheter. In certain embodiments, the cord comprises a memory metal wire.

In certain embodiments, attaching the cord to the first papillary muscle involves embedding an anchor attached to the cord in the first papillary muscle. In certain embodiments, attaching the cord to the first papillary muscle involves passing an end portion of the cord through the first papillary muscle and attaching the end portion to an anchor. The anchor may be, for example, a T-bar anchor. In certain embodiments, attaching the cord to the first papillary muscle comprises clamping a grasper component of the catheter on at least a portion of the first papillary muscle and introducing a needle into the first papillary muscle from the grasper component.

In certain embodiments, delivering the catheter into the ventricle of the heart is performed using a transcatheter procedure. For example, the transcatheter procedure may be a transfemoral procedure.

In some implementations, the present disclosure relates to a papillary muscle binding system comprising a cord configured to be attached to first and second papillary muscles disposed in a ventricle of a heart, the first and second papillary muscles being connected to first and second leaflets, respectively, of an atrioventricular heart valve, and a locker device configured to secure first and second end portions of the cord in a fixed relative position to form a loop attached to both the first and second papillary muscles. Implantation of the binding system can reduce prolapse of one or more of the first and second leaflets.

In certain embodiments, the locker device is a winch locker. For example, the winch locker may comprise a spool component configured to adjust a tension of the cord when wound. In certain embodiments, the locker device is a clamp. The cord and/or locker device may be adjustable. In certain embodiments, the papillary muscle binding system further comprises first and second anchors configured to be anchored to the first and second papillary muscles, respectively. For example, the first and second anchors may be T-bar anchors. In certain embodiments, the cord comprises a memory metal wire.

### BRIEF DESCRIPTION OF THE DRAWINGS

Various embodiments are depicted in the accompanying drawings for illustrative purposes, and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 provides a cross-sectional view of a human heart.

FIG. 2 provides a cross-sectional view of the left ventricle and left atrium of an example heart.

FIG. 3 provides a cross-sectional view of a heart experiencing mitral regurgitation.

FIG. 4 illustrates a cross-section of a heart having a papillary muscle binding system deployed therein according to one or more embodiments.

FIG. 5 shows a top view of a papillary muscle binding system in a deployed state in a ventricle of a heart according to one or more embodiments.

FIGS. 6-1 and 6-2 provide flow diagrams representing processes for binding papillary muscles according to one or more embodiments disclosed herein.

FIGS. 7-1 and 7-2 shows examples of various stages of the processes for binding papillary muscles as shown in FIGS. 6-1 and 6-2, respectively.

FIG. 8 is a flow diagram for a process for puncturing a papillary muscle using a grasper catheter according to one or more embodiments.

FIG. 9 shows examples of various stages of the process for puncturing a papillary muscle using a grasper catheter as shown in FIG. 8.

FIG. 10 illustrates a papillary muscle binding system according to one or more embodiments.

FIG. 11 illustrates a papillary muscle binding system according to one or more embodiments.

FIG. 12 illustrates a papillary muscle binding system according to one or more embodiments.

FIG. 13 illustrates a papillary muscle binding system according to one or more embodiments.

### DETAILED DESCRIPTION

The headings provided herein are for convenience only and do not necessarily affect the scope or meaning of the claimed invention.

Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### Overview

In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.).

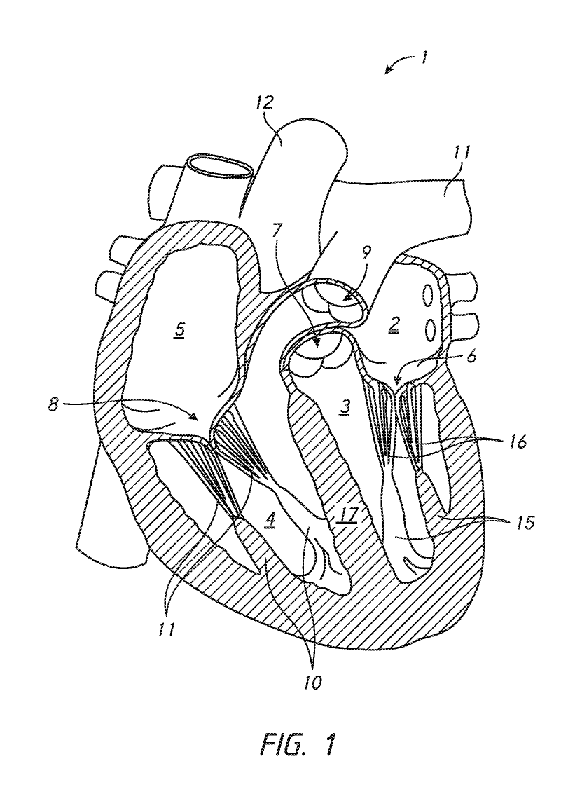

FIG. 1 illustrates an example representation of a heart 1 having various features relevant to certain embodiments of the present inventive disclosure. The heart 1 includes four chambers, namely the left atrium 2, the left ventricle 3, the right ventricle 4, and the right atrium 5. A wall of muscle 17, referred to as the septum, separates the left 2 and right 5 atria and the left 3 and right 4 ventricles. The heart 1 further includes four valves for aiding the circulation of blood therein, including the tricuspid valve 8, which separates the right atrium 5 from the right ventricle 4. The tricuspid valve 8 may generally have three cusps or leaflets and may generally close during ventricular contraction (i.e., systole) and open during ventricular expansion (i.e., diastole). The valves of the heart 1 further include the pulmonary valve 9, which separates the right ventricle 4 from the pulmonary artery 11, and may be configured to open during systole so that blood may be pumped toward the lungs, and close during diastole to prevent blood from leaking back into the heart from the pulmonary artery. The pulmonary valve 9 generally has three cusps/leaflets, wherein each one may have a crescent-type shape. The heart 1 further includes the mitral valve 6, which generally has two cusps/leaflets and separates the left atrium 2 from the left ventricle 3. The mitral valve 6 may generally be configured to open during diastole so that blood in the left atrium 2 can flow into the left ventricle 3, and advantageously close during diastole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

Heart valves may generally comprise a relatively dense fibrous ring, referred to herein as the annulus, as well as a plurality of leaflets or cusps attached to the annulus. Generally, the size and position of the leaflets or cusps may be such that when the heart contracts, the resulting increased blood pressure produced within the corresponding heart chamber forces the leaflets at least partially open to allow flow from the heart chamber. As the pressure in the heart chamber subsides, the pressure in the subsequent chamber or blood vessel may become dominant, and press back against the leaflets. As a result, the leaflets/cusps come in apposition to each other, thereby closing the flow passage.

The atrioventricular (i.e., mitral and tricuspid) heart valves may further comprise a collection of chordae tendineae and papillary muscles for securing the leaflets of the respective valves to promote and/or facilitate proper coaptation of the valve leaflets and prevent prolapse thereof. The papillary muscles, for example, may generally comprise finger-like projections from the ventricle wall.

With respect to the mitral valve 6, a normal mitral valve may comprise two leaflets (anterior and posterior) and two corresponding papillary muscles 15. The papillary muscles 15 originate in the left ventricle wall and project into the left ventricle 3. Generally, the anterior leaflet may cover approximately two-thirds of the valve annulus. Although the anterior leaflet covers a greater portion of the annulus, the posterior leaflet may comprise a larger surface area in certain anatomies. The valve leaflets of the mitral valve 6 may be prevented from prolapsing into the left atrium 2 by the action of the chordae tendineae 16 tendons connecting the valve leaflets to the papillary muscles 15. The relatively inelastic chordae tendineae 16 are attached at one end to the papillary muscles 15 and at the other to the valve leaflets; chordae tendineae from each of the papillary muscles 15 are attached to a respective leaflet of the mitral valve 6. Thus, when the left ventricle 3 contracts, the intraventricular pressure forces the valve to close, while the chordae tendineae 16 keep the leaflets coapting together and prevent the valve from opening in the wrong direction, thereby preventing blood to flow back to the left atrium 2. The various chords of the chordae tendineae may have different thicknesses, wherein relatively thinner chords are attached to the free leaflet margin, while relatively thicker chords (e.g., strut chords) are attached farther away from the free margin.

With respect to the tricuspid valve 8, the normal tricuspid valve may comprise three leaflets (two shown in FIG. 1) and three corresponding papillary muscles 10 (two shown in FIG. 1). The leaflets of the tricuspid valve may be referred to as the anterior, posterior and septal leaflets, respectively. The valve leaflets are connected to the papillary muscles by the chordae tendineae 11, which are disposed in the right ventricle 4 along with the papillary muscles 10. Although tricuspid valves are described herein as comprising three leaflets, it should be understood that tricuspid valves may occur with two or four leaflets in certain patients and/or conditions; the principles relating to papillary muscle binding and/or adjustment disclosed herein are applicable to atrioventricular valves having any number of leaflets and/or papillary muscles associated therewith. The right ventricular papillary muscles 10 originate in the right ventricle wall, and attach to the anterior, posterior and septal leaflets of the tricuspid valve, respectively, via the chordae tendineae 11. The papillary muscles 10 of the right ventricle 4 may have variable anatomy; the anterior papillary may generally be the most prominent of the papillary muscles. The papillary muscles 10 may serve to secure the leaflets of the tricuspid valve 8 to prevent prolapsing of the leaflets into the right atrium 5 during ventricular systole. Tricuspid regurgitation can be the result of papillary dysfunction or chordae rupture.

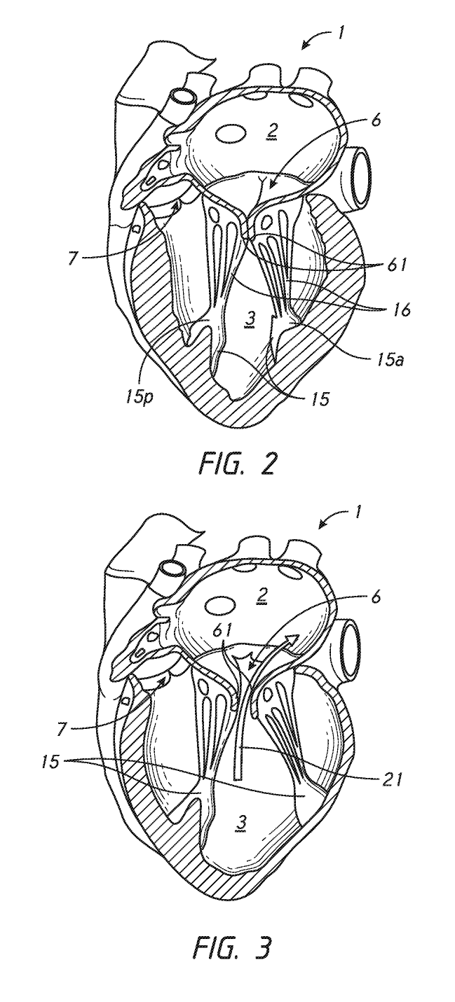

FIG. 2 provides a cross-sectional view of the left ventricle 3 and left atrium 2 of an example heart 1. The diagram of FIG. 2 shows the mitral valve 6, wherein the disposition of the valve 6, papillary muscles 15 and/or 16 may be illustrative as providing for proper coapting of the valve leaflets to advantageously at least partially prevent regurgitation and/or undesirable flow into the left atrium from the left ventricle 3 and vice versa. Although a mitral valve 6 is shown in FIG. 2 and various other figures provided herewith, and described herein in the context of certain embodiments of the present disclosure, it should be understood that papillary muscle binding and/or adjustment principles disclosed herein may be applicable with respect to any atrioventricular valve and associated anatomy (e.g., papillary muscles, chordae tendineae, ventricle wall, etc.), such as the tricuspid valve.

As described above, with respect to a healthy heart valve as shown in FIG. 2, the valve leaflets 61 may extend inward from the valve annulus and come together in the flow orifice to permit flow in the outflow direction (e.g., the downward direction in FIG. 2) and prevent backflow or regurgitation toward the inflow direction (e.g., the upward direction in FIG. 2). For example, during atrial systole, blood flows from the atria 2 to the ventricle 3 down the pressure gradient, resulting in the chordae tendineae 16 being relaxed due to the atrioventricular valve 6 being forced open. When the ventricle 3 contracts during ventricular systole, the increased blood pressures in both chambers may push the valve 6 closed, preventing backflow of blood into the atria 2. Due to the lower blood pressure in the atria compared to the ventricles, the valve leaflets may tend to be drawn toward the atria. The chordae tendineae 16 can serve to tether the leaflets and hold them in a closed position when they become tense during ventricular systole. The papillary muscles 15 provide structures in the ventricles for securing the chordae tendineae and therefore allowing the chordae tendineae to hold the leaflets in a closed position. The papillary muscles 15 may include an anterolateral papillary muscle 15a, which may be tethered to the posterior leaflet, for example, and a posteromedial papillary muscle 15p, which may be tethered to the anterior leaflet, for example. With respect to the state of the heart 1 shown in FIG. 2, the proper coaptation of the valve leaflets, which may be due in part to proper position of the papillary muscles 15, may advantageously result in mitral valve operation substantially free of leakage.

Heart valve disease represents a condition in which one or more of the valves of the heart fails to function properly. Diseased heart valves may be categorized as stenotic, wherein the valve does not open sufficiently to allow adequate forward flow of blood through the valve, and/or incompetent, wherein the valve does not close completely, causing excessive backward flow of blood through the valve when the valve is closed. In certain conditions, valve disease can be severely debilitating and even fatal if left untreated. With regard to incompetent heart valves, over time and/or due to various physiological conditions, the position of papillary muscles may become altered, thereby potentially contributing to valve regurgitation. For example, functional mitral valve regurgitation may be considered a disease of the left ventricle rather than of the mitral valve itself. functional mitral valve regurgitation can occur when the left ventricle of the heart is distorted or dilated, displacing the papillary muscles that support the two valve leaflets. For example, the valve leaflets may no longer come together to close the annulus, thereby resulting in blood flow back into the atrium. If left untreated, FMR can overload the heart and can lead to or accelerate heart failure. Moving or pulling the papillary muscles closer to their natural positions can potentially reduce occurrence of valve regurgitation.

FIG. 3 illustrates a cross-sectional view of a heart 1 experiencing functional mitral valve regurgitation flow 21, dilation of the left ventricle may cause changes in the position of the papillary muscles 15 that allow flow 21 back from the ventricle 3 to the atrium 2. Dilation of the left ventricle can be causes by any number of conditions, such as focal myocardial infarction, global ischemia of the myocardial tissue, or idiopathic dilated cardiomyopathy, resulting in alterations in the geometric relationship between papillary muscles and other components associated with the valve(s) that can cause valve regurgitation. Functional regurgitation may further be present even where the valve components may be normal pathologically, yet may be unable to function properly due to changes in the surrounding environment. Examples of such changes include geometric alterations of one or more heart chambers and/or decreases in myocardial contractility. In any case, the resultant volume overload that exists as a result of an insufficient valve may increase chamber wall stress, which may eventually result in a dilatory effect that causes papillary muscle alteration resulting in valve dysfunction and degraded cardiac efficiency.

Functional mitral valve regurgitation may occur when the left ventricle 3 of the heart 1 is distorted or dilated, displacing the papillary muscles 15 that support the two valve leaflets 61. The valve leaflets 61 therefore may no longer come together sufficiently to close the annulus and prevent blood flow back into the atrium 2. If left untreated, the functional mitral valve regurgitation experienced in the state shown in FIG. 3 may overload the heart 1 and can possibly lead to or accelerate heart failure. Solutions presented herein provide devices and methods for moving the papillary muscles 15 closer to their previous position, which may advantageously reduce the occurrence of mitral regurgitation. As shown in FIG. 3, the failure of the leaflets 61 of the mitral valve (or tricuspid valve) to come into a state of coaptation results in an opening between the mitral valve leaflets 61 during the systolic phase of the cardiac cycle, which allows the leakage flow 21 of fluid back up into the atrium 2. The papillary muscles 15 may be displaced due to dilation of the left ventricle 3, or due to one or more other conditions, as described above, which may contribute to the failure of the valve 6 to close properly. In addition to the unwanted flow in the outflow direction (e.g., the upward direction in FIG. 3), the failure of the valve leaflets 61 to coapt properly may result in unwanted backflow or regurgitation toward the inflow direction (e.g., the downward direction in FIG. 2) as well in certain conditions.

Certain embodiments disclosed herein provide solutions for incompetent heart valves that involve binding two or more papillary muscles in order to at least partially re-position and/or adjust the papillary muscles in an inward direction. Solutions presented herein may be used to at least partially change the position of one or more papillary muscles in order to reduce the occurrences and/or severity of regurgitation, such as mitral regurgitation.

Various techniques that suffer from certain drawbacks may be implemented for treating mitral valve dysfunction, including surgical repair or replacement of the diseased valve or medical management of the patient, which may be appropriate/effective primarily in early stages of mitral valve dysfunction, during which levels of regurgitation may be relatively low. For example, such medical management may generally focus on volume reductions, such as diuresis or afterload reducers, such as vasodilators, for example. Valve replacement operations may also be used to treat regurgitation from valve dysfunction. However, such operations can result in ventricular dysfunction or failure following surgery. Further limitations to valve replacement solutions may include the potential need for lifelong therapy with powerful anticoagulants in order to mitigate the thromboembolic potential of prosthetic valve implants. Moreover, in the case of biologically-derived devices, such as those used as mitral valve replacements, the long-term durability may be limited. Another commonly employed repair technique involves the use of annuloplasty rings to improve mitral valve function. An annuloplasty may be placed in the valve annulus and the tissue of the annulus sewn or otherwise secured to the ring. Annuloplasty rings can provide a reduction in the annular circumference and/or an increase in the leaflet coaptation area. However, annuloplasty rings may flatten the saddle-like shape of the valve and/or hinder the natural contraction of the valve annulus. In addition, various surgical techniques may be used to treat valve dysfunction. However, such techniques may suffer from various limitations, such as requiring opening the heart to gain direct access to the valve and the valve annulus. Therefore, cardiopulmonary bypass may be required, which may introduce additional morbidity and mortality to the surgical procedures. Additionally, for surgical procedures, it can be difficult or impossible to evaluate the efficacy of the repair prior to the conclusion of the operation.

Disclosed herein are devices and methods for treating valve dysfunction without the need for cardiopulmonary bypass and without requiring major remodeling of the dysfunctional valve. In particular, passive techniques to bind papillary muscles together to thereby change the shape and/or position of the papillary muscles are disclosed for reducing regurgitation while maintaining substantially normal leaflet anatomy. Further, various embodiments disclosed herein provide for the treatment of valve dysfunction that can be executed on a beating heart, thereby allowing for the ability to assess the efficacy of the papillary muscle re-positioning treatment and potentially implement modification thereto without the need for bypass support.

### Papillary Muscle Binding

Certain embodiments disclosed herein provide for systems, devices and methods for binding two or more papillary muscles in the left and/or right ventricles of a heart in order to improve valve coaptation during ventricular systole. For example, papillary adjustment devices are disclosed that may be implanted independently in one of the ventricles of the heart. Such devices may be introduced into the patient system through surgical or, advantageously, minimally-invasive means.

Papillary muscle binding methods and devices disclosed herein may include the use of one or more cords or wires for tying or holding two or more papillary muscles in a desirable relative position. The terms “cord” and “wire” are used herein according to their broad and ordinary meaning and may refer to any type or shape of cord or wire made of any suitable or desirable material, including any type of tie, line, rope, strand, cordage, tether, connector or cable. In certain embodiments, one or more papillary muscle binding cords may be used to pierce one or more papillary muscles in a ventricle of a heart, or otherwise be bonded, connected or attached thereto, wherein tightening or pulling together the cord(s) may enable the papillary muscles attached to or associated with the cord(s) to move closer together to potentially reduce regurgitation with respect to the valve associated with the papillary muscles. For example, the cord(s) may be used as sutures to puncture the papillary muscles in order to grasp/hold the papillary muscles in the desired position.

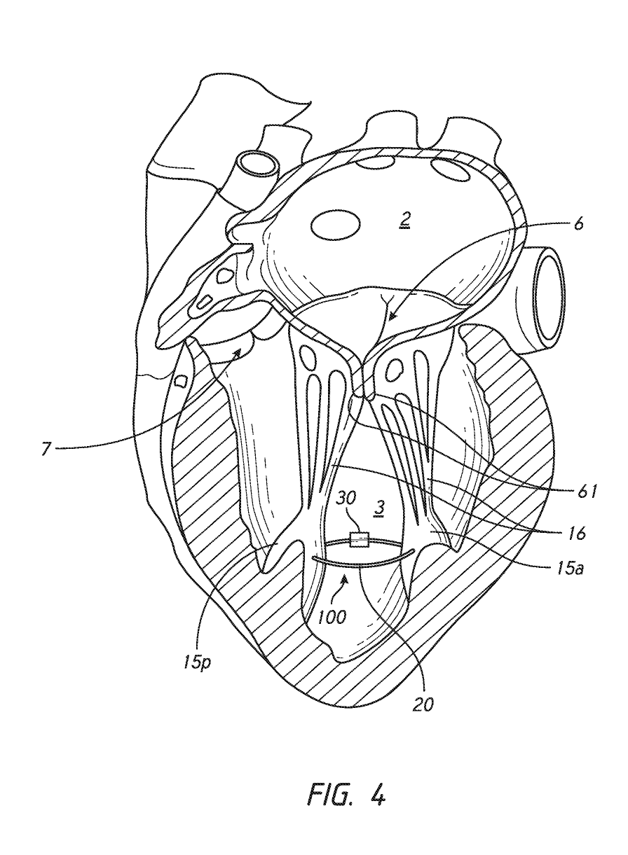

FIG. 4 illustrates a cross-section of a heart 1 having a papillary muscle binding system 100 deployed therein. The illustration of FIG. 4 shows a left ventricle 3 of the heart 1. Although certain disclosure herein is presented in the context of the left ventricle and associated anatomy (e.g., valves, papillary muscles, chordae tendineae, ventricle wall, etc.), it should be understood that the principles disclosed herein may be applicable in any ventricle of the heart (e.g., right ventricle) and associated anatomy (e.g., tricuspid valve, papillary muscles, chordae tendineae, ventricle wall, etc.). As described above, in a normal heart, the papillary muscles may contract during the heart cycle to assist in maintaining proper valve function. Reductions in, or failure of, the papillary muscle function can contribute to valve dysfunction and/or regurgitation, which may be caused by infarction at or near the papillary muscle, ischemia, or other causes, such as idiopathic dilated cardiomyopathy, for example.

FIG. 4 shows a papillary muscle binding cord 20 binding two papillary muscles 15p, 15a. The cord 20 may be coupled or attached to the papillary muscles 15p, 15a in any suitable or desirable manner. In certain embodiments, the cord 20 may provide a suture that punctures through at least part of the papillary muscle(s). The cord 20 may be held together to form a loop by a locking member 30, which may enable cinching and/or locking of two or more ends or portions of the cord 20 or cord(s). The tension in the cord 20 may cause the papillary muscles 15p, 15a to reposition inward, thereby lessening the traction of the chordae tendineae 16 on the corresponding leaflets 61 of the mitral valve, thereby resulting in improved coaptation of the mitral valve leaflets 61 during closure of the valve 6.

With respect to embodiments in which the papillary muscle binding system 100 is implemented in the right ventricle, the system 100 may serve to correct tricuspid regurgitation, which, similar to mitral regurgitation, involves a disorder in which the tricuspid valve does not close tight enough to prevent backflow through the valve. During tricuspid regurgitation, blood may flow backward into the right atrium when the right ventricle contracts. Such tricuspid valve dysfunction may result from the increase in size of the right ventricle. For example, enlargement or dilation of the right ventricle may result from high blood pressure in the arteries of the lungs, or from other heart problems, such as poor squeezing of the left side of the heart, or from problems with the opening or closing of another one of the heart valves.

The cord 20 and associated assembly/system may be introduced into the ventricle 3 and implanted using a delivery system, which may include a catheter for navigating the papillary muscle binding system to the desired position and performing the implantation. For example, the papillary muscle binding system 100 may be inserted non-surgically in, for example, a transcatheter procedure (e.g., transfemoral, transseptal, transapical, etc.), wherein the system 100 is inserted into the left ventricle 3 from the aorta 12 through the aortic valve 7. With respect to right ventricle papillary muscle adjustment, the binding system 100 may be inserted into the right ventricle from the pulmonary artery through the pulmonary valve.

In certain embodiments, the desired attachment/puncture position of the binding system 100 to the papillary muscles and/or the tension of the cord(s) 20 may be determined based on the resulting movement of the papillary muscles and/or the reduction in mitral (or tricuspid) regurgitation performance from implementation of the binding system 100, which may be observed using echocardiography or other means. When it is determined that the result is satisfactory, the cord 20 may be locked and detached from the delivery system at the desired position/tension.

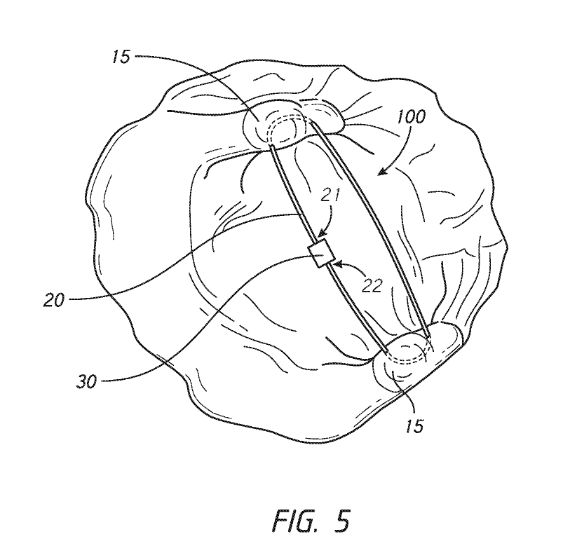

FIG. 5 shows a top view of a papillary muscle binding system 100 in a deployed state in a ventricle of a heart, wherein the binding system 100 is configured to maintain the papillary muscles at a desirable proximity to one another in accordance with embodiments disclosed herein. The binding system 100 includes a cinching locker 30 and main cord or suture 20, which may be threaded through at least part of the papillary muscles 15.

The binding cord 20 may be fixed to the papillary muscles 15 in any suitable or desirable way. For example, the cord 20 may be sutured to the papillary muscles 15, or anchored using a barb-like hook or corkscrew structure, or other type of anchor that may be attached to, or embedded in, the tissue of the papillary muscles. Tension of the cord 20 can be cinched to a desired degree in place using echocardiography to observe whether reduced mitral regurgitation and/or desired leaflet seating is produced thereby.

The locker member 30 may serve to cinch/hold the cord ends in a locked position to provide desirable tension in the cord 20. The locker/cincher 30 may be implemented in any suitable or desirable way or configuration. In certain embodiments, the locker member 30 may comprise a locking bid-tube structure with flaps that close down over portions or ends of the cord to lock them in position. Alternatively, the locker member 30 may comprise a winch device that may be configured to wind up one or more ends or portions of the cord 20 about a spool component or the like. For example, the locker 30 may comprise an external cylinder and/or an inner cylinder configured to rotate, thereby pulling the cord 20 to cinch and to lock the cord at the desired tension. The locker 30 may clamp or otherwise secure first and second portions (21, 22) of the cord in a relative fixed position. For example, the first and second portions (21, 22) may correspond to separate ends of the cord 20 when the binding system 100 has been deployed in the ventricle of the heart.

By observing the performance and/or physical structure of the atrioventricular valve and/or associated anatomy (e.g., papillary muscle(s)) during the implantation process, papillary muscle binding methods disclosed here may advantageously allow for substantially immediate or real-time adjustment of the tension and/or attachment/suture position of the cord 20 while the delivery system is locally disposed and available for adjustment operations. Once the desired configuration of the papillary muscle binding system 100 has been achieved and the binding system 100 has been released from the delivery system, the binding system 100 may continually provide desirable inward repositioning of the papillary muscles 15 on an on-going operational basis to reduce valve regurgitation, as explained above.

Although cords/wires are disclosed herein in the context of certain embodiments of papillary muscle binding systems, it should be understood that papillary muscle binding systems in accordance with the present disclosure may comprise any desirable shape, size or type of device, and may include one or more straps, bands, or other types of devices/forms.

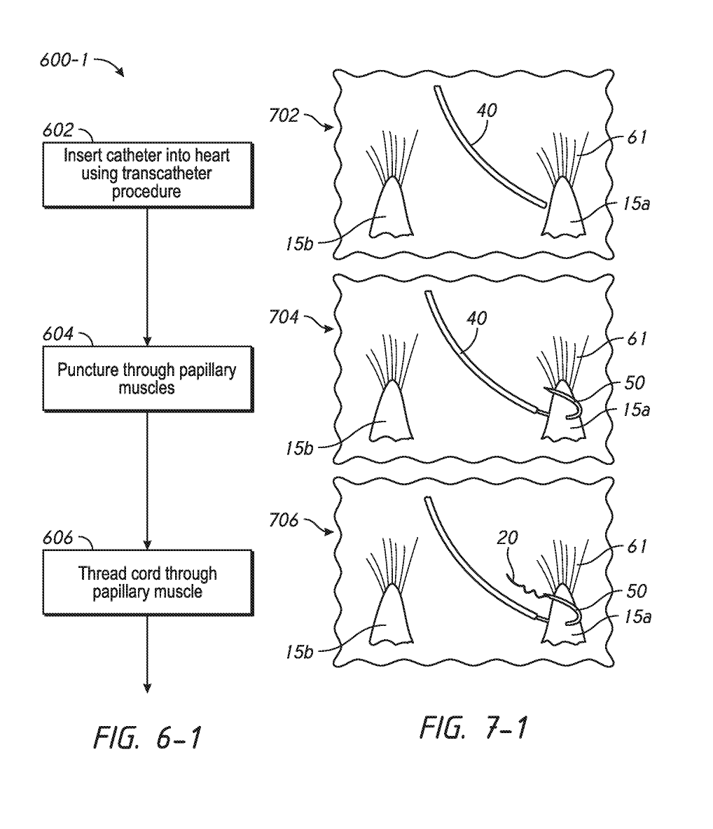

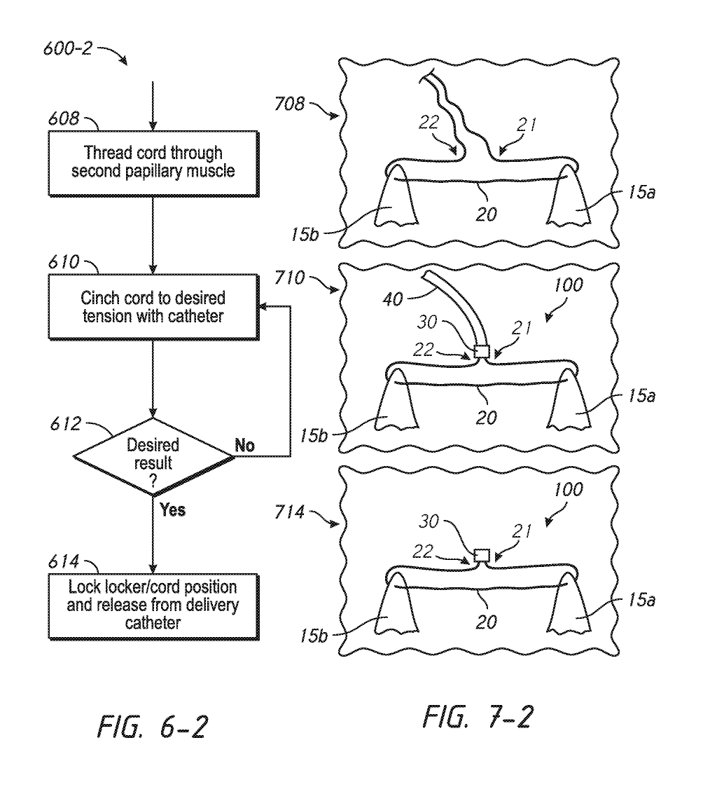

FIGS. 6-1 and 6-2 provide flow diagrams representing processes (600-1, 600-2) for binding papillary muscles according to one or more embodiments disclosed herein. FIGS. 7-1 and 7-2 shows examples of various stages of the processes 600-1 and 600-2 for binding papillary muscles as shown in FIGS. 6-1 and 6-2, respectively. The processes 600-1, 600-2 describe certain steps and/or operations for implanting and/or locking/cinching a binding cord system in accordance with certain embodiments disclosed herein.

At block 602, the process 600-1 involves inserting a catheter delivery system 40 into a ventricle of the heart, such as the left ventricle, using a transcatheter procedure. For example, the catheter 40 may be delivered using a transfemoral, transendocardial, transcoronary, transseptal, transapical, or other approach. Alternatively, the catheter 40 may be introduced into the desired location during an open-chest surgical procedure, or using other surgical or non-surgical techniques known in the art. In accordance with certain embodiments, the catheter 40 may be brought into proximity with one of the papillary muscles (e.g., papillary muscle 15a) of the left (or right) ventricle. The reference identifiers 15a and 15b are used in connection with FIGS. 7-1 and 7-2 arbitrarily, and each may represent any papillary muscle of either the left or right ventricle of the heart.

At block 604, the process 600-1 involves puncturing through the papillary muscle 15a that is proximate the distal end of the catheter 40, or otherwise creating an attachment to the papillary muscle, such as by wrapping a cord or other component around the papillary muscle or embedding an anchor in tissue of the papillary muscle. Puncturing the papillary muscle 15a may be achieved using a puncturing tool 50, such as a needle, which may be delivered using the catheter 40. The catheter 40 may advantageously be designed to attach to the papillary muscle 15a in a stable and/or predictable manner. In certain embodiments, the papillary muscle 15a is punctured without the use of a needle.

In certain embodiments, the catheter 40 achieves attachment to the papillary muscle 15a through the use of a flexible catheter having a distal end shape that is configured to open to clamp on or encircle the papillary muscle. A puncturing tool, such as a flexible needle, may be introduced from the end of the catheter 40 to puncture the papillary muscle 15a and possibly to thread a suture/cord 20 therethrough. In certain embodiments, the catheter 40 comprises a vacuum catheter having a distal suction nipple that is configured to attach to the papillary muscle and enable needle puncturing from the catheter. The catheter 40 may be introduced into the ventricle in any suitable or desirable manner, such as through a transfemoral procedure through the aortic valve, or alternatively through a transapical procedure.

At block 606, the process 600-1 involves threading a cord 20 or other form through the puncturing tool 50 to thereby pass through the papillary muscle 15a. Although the cord 20 is shown in state 706 of FIG. 7-1 as being threaded through the papillary muscle 15a through the puncturing tool 50, it should be understood that the process step 604 may involve attaching or otherwise associating the cord to the papillary muscle 15a without running the cord 20 through the papillary muscle, such as by using an anchor or other attachment means. In certain embodiments, the cord 20 comprises a wire (e.g. Nitinol memory metal wire) that can be pushed and/or pulled through the papillary muscle to produce the papillary muscle binding configuration illustrated and/or described in connection with one or more embodiments.

At block 608, the process 600-2 involves threading the cord 20 through another papillary muscle 15b. Similarly to step 606, in certain embodiments, the cord 20 may not be threaded through the papillary muscle 15b, but may be otherwise attached to or wrapped around the papillary muscle 16b in such a manner as to allow the cord 20 to exert displacement force on the papillary muscle by pulling on the cord 20. The cord 20 may be threaded or attached to the papillary muscle 15b in any of the ways or using any of the mechanisms described above with respect to process step 606 and state 706.

With the cord 20 attached to both papillary muscles 15a, 15b (or to three papillary muscles in certain embodiments), at block 610, the process 600 involves cinching the cord 20 to a desired tension. Such cinching of the cord 20 may be performed at least in part using the catheter 40. For example, the cord 20 may be used as a rail to guide the cinching catheter 40 to a desired tension. The cinching catheter 40 may at least partially control the distance between the papillary muscles 15a, 15b for achieving the desired reshaping of the valve and/or associated anatomy. In certain embodiments, the distal end of the cord 20 may be fed into a rail guide or other structure, such as the locker 30, to allow the cord to be cinched by the catheter 40 and/or locker 30. For example, the distal end of the cord 20 may be fed up into the catheter a desirable distance to allow for cinching of the cord. Therefore, in certain embodiments, the distal end of the cord 20 may be directed out of the catheter 40, through a first papillary muscle, through a second papillary muscle, and back into the catheter 40 and/or into the locker 30. The locker 30 may be designed to be slid through the catheter and over the distal end of the cord and an intermediate portion 21 of the cord, wherein the intermediate portion 21 of the cord 20 is a portion of the cord that is desired to be clamped or fixed to provide a locking position for the binding system 100.

The degree to which the cord 20 is tightened may be determined by the resulting movement of the papillary muscles and/or reduction in regurgitation. At block 612, the process 600-2 involves evaluating papillary muscle position and/or valve regurgitation resulting from the binding of the papillary muscles to determine the effectiveness of the device to determining whether the evaluated position of the papillary muscles and/or regurgitation performance evaluated/observed is satisfactory or desirable. If not, the process 600-2 returns to block 610, where the cord may be further tightened or loosened to thereby further alter the effect of the cord 20 on the papillary muscles 15a, 15b. In certain embodiments, the operator may use echocardiography or any other suitable means to observe the movement of the valve leaflets, such as in real time. The results of the cord 20 tensioning may be observed continuously or at selected intervals to determine when the papillary muscles have been repositioned sufficiently to provide a desired improvement in closure of the valve during the phase of the cardiac cycle associated with closure of the relevant valve (e.g., during systole in the case of a mitral valve). Therefore, the processes 600-1, 600-2 and/or other processes, devices and systems disclosed herein may advantageously provide a tunable papillary muscle binding system, which may be tuned while monitoring the effect of the system, such as through the use of echo or other visualization guidance.

If the desired result is achieved as determined at block 612, the process 600-2 continues to block 614, where the cord may be locked in its desired position using a locker device 30, which may cinch down on one or more ends or portions of the cord and thereby hold the cord 20 in a fixed position. The catheter 40 may be detached or cut from the binding cord 20 to leave the final implant 700 as configured with the locker 30 holding the cord 20 at the desired length/position.

The locker 30 may hold first and second points or portions of the cord 20 in a fixed relative position. Releasing the cord 20 from the deliver catheter 40 may involve cutting one or more portions of the cord 20 at or near the locker 30. In certain embodiments, the locker 30 may be used to cut/sever the cord 20 to allow for the cord to be released from the catheter. In certain embodiments, releasing the cord 20 from the catheter 40 involves withdrawing the cord 20 from the catheter. In certain embodiments, once the binding system 100 has been released from the catheter 40, the locker may hold in a relative fixed position the distal ends of the cord 20 formed when intermediate portions 21 and 22 of the cord 20 are clamped or fixed by the locker 30.

In certain embodiments, the processes 600-1, 600-2 may advantageously provide mechanisms for treating valve regurgitation (e.g., functional mitral valve regurgitation) without the need for dedicated anchors, such as through the use of a cord and locker assembly, as shown in FIGS. 7-1 and 7-2. In addition, papillary muscle reshaping may be achieved through mere puncturing of the papillary muscles without additional puncturing of the myocardium tissue of the heart and into the pericardium.

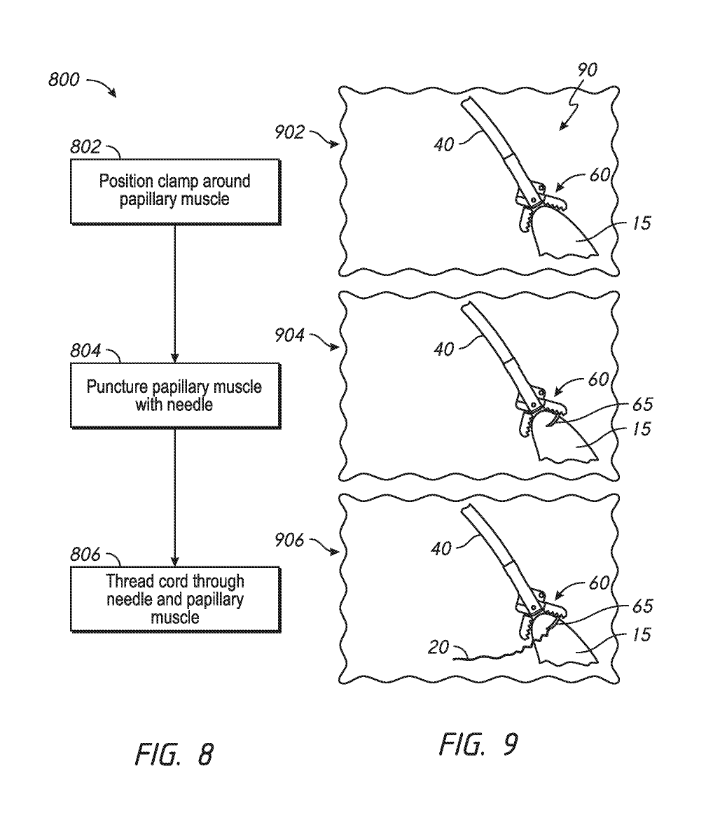

In accordance with papillary muscle binding systems and methods disclosed herein, any suitable or desirable form or type of catheter component may be used to grasp or attach to the papillary muscles. For example, certain embodiments implement grasper catheter systems including a flexible catheter that grasps the papillary muscle. FIG. 8 is a flow diagram for a process 800 for puncturing a papillary muscle 15 using a grasper catheter 90 having a catheter component 40 and a distal grasper component 60. FIG. 9 shows examples of various stages of the process 800 for puncturing a papillary muscle using a grasper catheter as shown in FIG. 8.

The grasper catheter 90 may be similar in certain respects to flexible biopsy forceps, which may be used for endoscopy procedures or the like. At block 802, the process 800 involves positioning a clamp of the grasper 60 around a papillary muscle 15, as shown at state 902 of FIG. 9. At block 804, the process 800 involves puncturing the papillary muscle 15 with a needle 65, as shown in at state 904 of FIG. 9. The needle 65 may be a flexible curved needle in certain embodiments, and may be introduced from the grasper portion 60 of the grasper catheter 90. At block 806, the process 800 involves threading a cord, such as a wire suture, through the needle and papillary muscle.

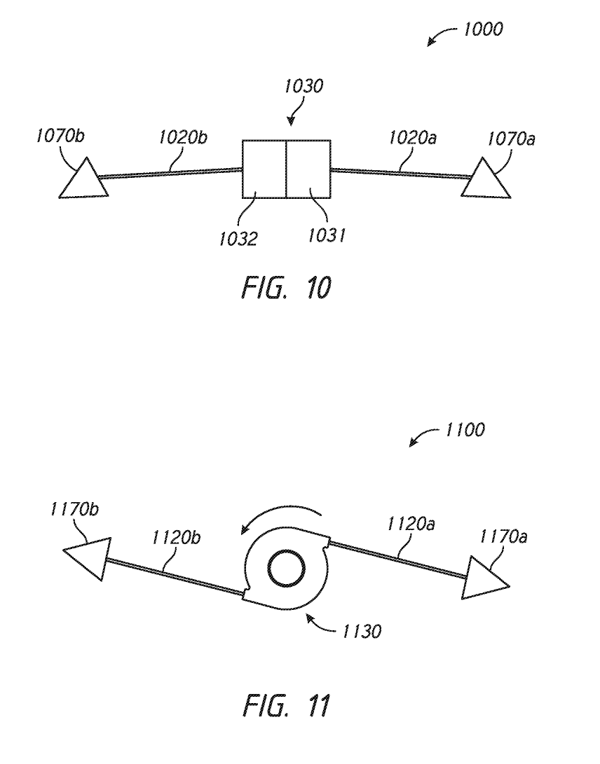

Papillary muscle binding systems according to embodiments disclosed herein may have any suitable or desirable form or configuration within the scope of the present disclosure. For example, in a final deployed state, a papillary muscle binding system may include various types and/or configurations of cords, anchors, and/or lockers. FIG. 10 illustrates a papillary muscle binding system 1000 including one or more cords or cord portions 1020a, 1020b coupled at distal ends to anchor members 1070a, 1070b, respectively. The system 1000 includes a locker device 1030, which may comprise a plurality of flap or clamp members that are configured to be closed down over one or more cord portions or ends to thereby secure the cord(s) in a fixed position relative to the locker 1030.

FIG. 11 illustrates a papillary muscle binding system 1100 including one or more cords or cord portions 1120a, 1120b coupled at distal ends to anchor members 1170a, 1170b, respectively. The system 1000 includes a locker device 1130, which may comprise a winch mechanism. For example, the locker 1130 may comprise one or more of an external cylinder and an inner cylinder configured to rotate to wind the cord(s) to shorten/tighten them. The locker 1130 may further be configured to lock in a desired position.

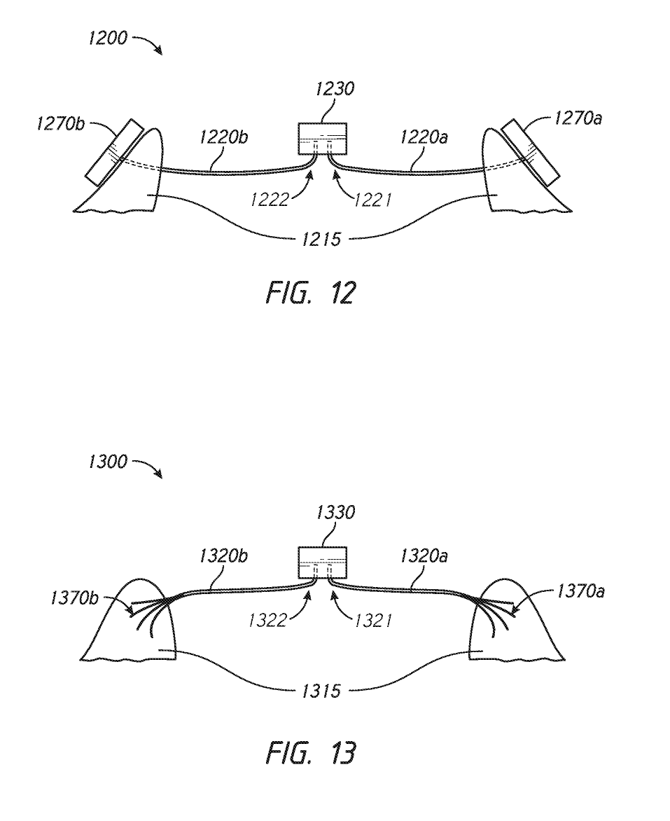

FIG. 12 illustrates a papillary muscle binding system 1200 including one or more cords or cord portions 1220a, 1220b coupled at distal ends to anchor members 1270a, 1270b, respectively. The anchors 1270a, 1270b may comprise T-bars that may be disposed at least partially behind the papillary muscles 1215 to allow for the cord(s) 1220a, 1220b to exert inward pulling force on the papillary muscles 1215. The system 1200 includes a locker device 1230, which may comprise any form or type in accordance with the present disclosure. The locker device 1230 may be configured to secure first and second portion (1221, 1222) of the respective cords (1220a, 1220b) in a fixed relative position.

FIG. 13 illustrates a papillary muscle binding system 1300 including one or more cords or cord portions 1320a, 1320b coupled at distal ends to anchor members 1370a, 1370b, respectively. The anchors 1370a, 1370b may comprise puncturing features that may be deployed into the papillary muscles 1315 to attach the cord(s) thereto and allow for the cord(s) 1320a, 1320b to exert inward pulling force on the papillary muscles 1315. The system 1300 includes a locker device 1330, which may comprise any form or type in accordance with the present disclosure. The locker device 1330 may be configured to secure first and second portion (1321, 1322) of the respective cords (1320a, 1320b) in a fixed relative position.

### Additional Embodiments

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes. Moreover, in certain embodiments, acts or events may be performed concurrently, e.g., through multi-threaded processing, interrupt processing, or via multiple processors or processor cores, rather than sequentially.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

What is claimed is:

1. A method for treating a heart valve, said method comprising:
delivering a catheter into a ventricle of a heart;
attaching a cord to a first papillary muscle disposed in the ventricle, the first papillary muscle being connected to a first leaflet of an atrioventricular heart valve;
attaching the cord to a second papillary muscle, the second papillary muscle being connected to a second leaflet of the heart valve;
securing first and second portions of the cord in a fixed relative position to form a loop attached to both the first and second papillary muscles; and
releasing the cord from the catheter.

2. The method of claim 1, wherein performing the method improves at least one of prolapse of the first and second leaflets and regurgitation of the heart valve.

3. The method of claim 1, wherein said attaching the cord to the first papillary muscle comprises puncturing the first papillary muscle and threading a distal end of the cord through at least a portion of the first papillary muscle.

4. The method of claim 3, wherein said puncturing comprises introducing a flexible needle from the catheter and puncturing the first papillary muscle using the flexible needle.

5. The method of claim 1 wherein said securing the first and second portions of the cord in a fixed relative position comprises:
introducing the first and second portions of the cord into a locker device; and
locking the locker device to secure the first and second portions of the cord.

6. The method of claim 5, wherein the locker device is a winch locker.

7. The method of claim 6, further comprising winding one or more of the first and second portions of the cord on a spool component of the winch locker to achieve a desired tension in the cord.

8. The method of claim 5, wherein the locker device is a clamp, wherein said locking the locker device comprises closing first and second portions of the clamp over the first and second portions of the cord.

9. The method of claim 1, further comprising:
after said attaching the cord to the second papillary muscle, determining whether function of the heart valve is adequate; and
when it is determined that the function of the heart valve is not adequate, adjusting a tension of the cord prior to said releasing the cord from the catheter.

10. The method of claim 9, wherein said determining whether function of the heart valve is adequate is performed at least part using echocardiography.

11. The method of claim 9, wherein said determining whether function of the heart valve is adequate comprises determining whether the first papillary muscle has moved to a desirable position.

12. The method of claim 9, wherein said determining whether function of the heart valve is adequate comprises determining whether regurgitation of the heart valve has been sufficiently reduced.

13. The method of claim 9, wherein said determining whether function of the heart valve is adequate comprises determining whether a position of the first or second leaflet has moved to a desirable position.

14. The method of claim 1, further comprising adjusting a tension of the cord prior to said securing the first and second portions of the cord.

15. The method of claim 14, wherein said adjusting the tension in the cord comprises cinching the cord using a portion of the catheter.

16. The method of claim 1, wherein the cord comprises a memory metal wire.

17. The method of claim 1, wherein said attaching the cord to the first papillary muscle involves embedding an anchor attached to the cord in the first papillary muscle.

18. The method of claim 1, wherein said attaching the cord to the first papillary muscle involves passing an end portion of the cord through the first papillary muscle and attaching the end portion to an anchor.

19. The method of claim 18, wherein the anchor is a T-bar anchor.

20. The method of claim 1, wherein said attaching the cord to the first papillary muscle comprises clamping a grasper component of the catheter on at least a portion of the first papillary muscle and introducing a needle into the first papillary muscle from the grasper component.

21. The method of claim 1, wherein said delivering the catheter into the ventricle of the heart is performed using a transcatheter procedure.

22. The method of claim 21, wherein the transcatheter procedure is a transfemoral procedure.

23. The method of claim 1, wherein the ventricle is a left ventricle of the heart and the heart valve is a mitral valve.

24. A papillary muscle binding system comprising:
a cord configured to be attached to first and second papillary muscles disposed in a ventricle of a heart, the first and second papillary muscles being connected to first and second leaflets, respectively, of an atrioventricular heart valve; and
a locker device configured to secure first and second end portions of the cord in a fixed relative position to form a loop attached to both the first and second papillary muscles.

25. The papillary muscle binding system of claim 24, wherein implantation of the binding system reduces prolapse of one or more of the first and second leaflets.

26. The papillary muscle binding system of claim 24, wherein the locker device is a winch locker.

27. The papillary muscle binding system of claim 26, wherein the winch locker comprises a spool component configured to adjust a tension of the cord when wound.

28. The papillary muscle binding system of claim 24, wherein the locker device is a clamp.

29. The papillary muscle binding system of claim 24, wherein the cord and locker device are adjustable.

30. The papillary muscle binding system of claim 24, further comprising first and second anchors configured to be anchored to the first and second papillary muscles, respectively.

31. The papillary muscle binding system of claim 30, wherein the first and second anchors are T-bar anchors.

32. The papillary muscle binding system of claim 24, wherein the cord comprises a memory metal wire.
